(19) United States
(12) Patent Application Publication
(10) Pub. No.: US 2006/0110765 A1
(43) Pub. Date: May 25, 2006

(54) Detection of nucleic acid variation by cleavage-amplification (CleavAmp) method

(76) Inventor: Xiao Bing Wang, Lutherville, MD (US)

Correspondence Address: MERCHANT & GOULD PC, P.O. BOX 2903, MINNEAPOLIS, MN, 55402-0903, US

(21) Appl. No.: 11/282,491

(22) Filed: Nov. 18, 2005

Related U.S. Application Data
(60) Provisional application No. 60/635,568, filed on Nov. 23, 2004.

(51) Int. Cl. C12Q 1/68; C12P 19/34

(52) U.S. Cl. 435/6; 435/91.2

(57) ABSTRACT

Methods and compositions for detecting nucleic acid polymorphisms are provided.

### CROSS-REFERENCE TO RELATED APPLICATION

This application claims benefit of and priority to U.S. Provisional Patent Application No. 60/635,568 filed on Nov. 23, 2004, and where permissible is incorporated by reference in its entirety.

### BACKGROUND

1. Technical Field

This disclosure is generally directed to methods and compositions for detecting nucleic acids, in particular nucleic acids having one or more specific nucleotides at a specific location.

2. Related Art

Genetic mutations can cause severe biological disorders such as cancer and inherited diseases. Detection of mutations can help the early diagnosis of genetic disorders and provide individualized information for drug treatment.

Non-sequencing methods using mismatch repair enzymes to detect nucleic acid variation are known (U.S. Pat. Nos. 5,698,400; 5,958,692; 5,217,863; 6,455,249; 6,110,684; and 5,891,629). These methods generally include: 1) hybridizing a probe to a target nucleic acid; 2) cleaving a mismatch between the probe and the target nucleic acid with an enzyme or chemicals; and 3) detecting the cleaved fragment. One disadvantage of these methods is low sensitivity because the detection is limited to detecting actually cleaved fragments. When a sample contains low copies of a target nucleic acid, for example a variation allele, the target nucleic acid is difficult to detect even using a large amount of target nucleic acid for the cleavage reaction. Other methods use a PCR amplified product for the cleavage. However, the problem of pre-PCR amplification is the non-selective amplification of nucleic acids. When a sample is dominated by wild type alleles, amplification will typically create more wild type copies than the variation copies. This disproportional amplification further reduces the sensitivity of the detection.

### SUMMARY

One aspect of the disclosure provides a method to detect a nucleotide base variation in a nucleic acid comprising (1) preparing a gene specific nucleic acid probe (probe) with non-extendable 3′ end; (2) hybridizing the probe to a target nucleic acid to form a duplex; (3) exposing the duplex to a cleavage enzyme or chemicals, wherein the enzyme or chemicals are able to recognize and cleave a structure resulting from a mismatch between the probe and the target nucleic acid; (4) cleaving the structure resulting from the mismatch to remove the non-extendable 3′ end from the probe and generate a new extendable 3′ end on the probe and optionally, on the target nucleic acid; (5) using the cleaved probe or target nucleic acid as a primer or/and template for selectively amplification by primer based or polymerase promoter based amplification methods; and (6) detecting amplified nucleic acid product, wherein the amplified product indicates the presence of a sequence variation or polymorphism in the target nucleic acid.

Another aspect provides a method for detecting a polymorphism in a polynucleotide including (1) annealing a probe to a polynucleotide to a region of the polynucleotide suspected of containing a polymorphism to form a complex, wherein the probe comprises a non-extendable 3′ end and is not complementary to the polymorphism; (2) contacting the complex with an enzyme or chemical that cleaves the probe and the polynucleotide at a region of mismatch between the probe and the polynucleotide to produce a probe with an extendible 3′ end; (3) adding an artificial template, wherein the cleaved probe acts as a primer for amplifying the artificial template; and (4) amplifying the artificial template, wherein the presence of an amplified product indicates the presence of the polymorphism.

Aspects of the disclosed subject matter provide methods that pre cleave variant alleles and then selectively amplify the cleaved variant allele without amplification of the wild type allele. This feature increases detection sensitivity and allows detection of a low copy number of the variant allele in a mixed sample containing high percentage of wild type allele.

### BRIEF DESCRIPTION OF THE DRAWINGS

FIGS. 1A and B show a schematic drawing of gene specific probe, artificial template, and the adapter primer for an exemplary cleavage-amplification system.

FIG. 2 show an exemplary method for cleavage-amplification detection.

FIGS. 3A-C show exemplary methods of amplification by RNA polymerase promoter based amplification.

FIG. 4 shows a schematic drawing of probe design for amplification of cleavage product by using real time PCR method.

FIG. 5 shows a gel with amplification products from an exemplary method.

FIG. 6 shows a gel with amplification products from another exemplary method.

### DETAILED DESCRIPTION

Definitions

As used herein, the terms “nucleic acid” and “polynucleotide” are interchangeable and refer to any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

The terms nucleic acid, polynucleotide, and nucleotide also specifically include nucleic acids composed of bases other than the five biologically occurring bases (adenine, guanine, thymine, cytosine and uracil). For example, a polynucleotide of the invention might contain at least one modified base moiety which is selected from the group including but not limited to 5-fluorouracil, 5-bromouracil, 5-chlorouracil, 5 -iodouracil, hypoxanthine, xanthine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acidmethylester, 3-(3-amino-3-N-2-carboxypropyl) uracil, (acp3)w, and 2,6-diaminopurine.

Furthermore, a polynucleotide of the invention may comprise at least one modified sugar moiety selected from the group including but not limited to arabinose, 2-fluoroarabinose, xylulose, and hexose. It is not intended that the present invention be limited by the source of the polynucleotide. The polynucleotide can be from a human or non-human mammal, or any other organism, or derived from any recombinant source, synthesized in vitro or by chemical synthesis. The polynucleotide may be DNA, RNA, cDNA, DNA-RNA, peptide nucleic acid (PNA), a hybrid or any mixture of the same, and may exist in a double-stranded, single-stranded or partially double-stranded form. The nucleic acids of the invention include both nucleic acids and fragments thereof, in purified or unpurified forms, including genes, chromosomes, plasmids, the genomes of biological material such as microorganisms, e.g., bacteria, yeasts, viruses, viroids, molds, fungi, plants, animals, humans, and the like.

The nucleic acid can be only a minor fraction of a complex mixture such as a biological sample. The nucleic acid can be obtained from a biological sample by procedures well known in the art.

A polynucleotide of the present invention can be derivitized or modified, for example, for the purpose of detection, by biotinylation, amine modification, alkylation, or other like modification. In some circumstances, for example where increased nuclease stability is desired, the invention can employ nucleic acids having modified internucleoside linkages. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide, dimethylenesulfoxide, dimethylene-sulfone, 2′-O-alkyl, and 2′-deoxy-2′-fluoro phosphorothioate internucleoside linkages are well known in the art (see, Uhlman et al., 1990, Chem. Rev. 90:543-584; Schneider et al. 1990, Tetrahedron Lett. 31:335, and references cited therein).

The term “oligonucleotide” refers to a relatively short, single stranded polynucleotide, usually of synthetic origin. An oligonucleotide typically comprises a sequence that is 8 to 100 nucleotides, preferably, 20 to 80 nucleotides, and more preferably, 30 to 60 nucleotides in length. Various techniques can be employed for preparing an oligonucleotide utilized in the present invention. Such an oligonucleotide can be obtained by biological synthesis or by chemical synthesis. For short sequences (up to about 100 nucleotides) chemical synthesis will frequently be more economical compared to biological synthesis. In addition to economy, chemical synthesis provides a convenient way of incorporating low molecular weight compounds and/or modified bases during synthesis. Furthermore, chemical synthesis is very flexible in the choice of length and region of the target polynucleotide binding sequence. The oligonucleotide can be synthesized by standard methods such as those used in commercial automated nucleic acid synthesizers. Chemical synthesis of DNA on a suitably modified glass or resin can result in DNA covalently attached to the surface. This may offer advantages in washing and sample handling. For longer sequences standard replication methods employed in molecular biology can be used such as the use of M13 for single stranded DNA as described by J. Messing, 1983, Methods Enzymol. 101:20-78. Other methods of oligonucleotide synthesis include phosphotriester and phosphodiester methods (Narang et al., 1979, Meth. Enzymol. 68:90) and synthesis on a support (Beaucage et al., 1981, Tetrahedron Letters 22:1859-1862) as well as phosphoramidate synthesis, Caruthers et al., 1988, Meth. Enzymol. 154:287-314, and others described in “Synthesis and Applications of DNA and RNA,” S. A. Narang, editor, Academic Press, New York, 1987, and the references contained therein.

An oligonucleotide “primer” can be employed in a chain extension reaction with a polynucleotide template such as in, for example, the amplification of a nucleic acid. The oligonucleotide primer is usually a synthetic oligonucleotide that is single stranded, containing a hybridizable sequence at or near its 3′-end that is capable of hybridizing with a defined sequence of the target or reference polynucleotide. Normally, the hybridizable sequence of the oligonucleotide primer has at least 90%, preferably 95%, most preferably 100%, complementarity to a defined sequence or primer binding site. In certain embodiments of the invention, the sequence of a primer can vary from ideal complementarity to introduce mutations into resulting amplicons, as discussed below. The number of nucleotides in the hybridizable sequence of an oligonucleotide primer should be such that stringency conditions used to hybridize the oligonucleotide primer will prevent excessive random non-specific hybridization. Usually, the number of nucleotides in the hybridizable sequence of the oligonucleotide primer will be at least ten nucleotides, preferably at least 15 nucleotides and, preferably 20 to 50, nucleotides. In addition, the primer may have a sequence at its 5′-end that does not hybridize to the target or reference polynucleotides that can have 1 to 60 nucleotides, 5 to 30 nucleotides or, preferably, 8 to 30 nucleotides.

The term “sample” refers to a material suspected of containing a nucleic acid of interest. Such samples include biological fluids such as blood, serum, plasma, sputum, lymphatic fluid, semen, vaginal mucus, feces, urine, spinal fluid, and the like; biological tissue such as hair and skin; and so forth. Other samples include cell cultures and the like, plants, food, forensic samples such as paper, fabrics and scrapings, water, sewage, medicinals, etc. When necessary, the sample may be pretreated with reagents to liquefy the sample and/or release the nucleic acids from binding substances. Such pretreatments are well known in the art.

The term “amplification,” as applied to nucleic acids refers to any method that results in the formation of one or more copies of a nucleic acid, where preferably the amplification is exponential. One such method for enzymatic amplification of specific sequences of DNA is known as the polymerase chain reaction (PCR), as described by Saiki et al., 1986, Science 230:1350-1354. Primers used in PCR can vary in length from about 10 to 50 or more nucleotides, and are typically selected to be at least about 15 nucleotides to ensure sufficient specificity. The double stranded fragment that is produced is called an “amplicon” and may vary in length from as few as about 30 nucleotides to 20,000 or more. The term “chain extension” refers to the extension of a 3′-end of a polynucleotide by the addition of nucleotides or bases. Chain extension relevant to the present invention is generally template dependent, that is, the appended nucleotides are determined by the sequence of a template nucleic acid to which the extending chain is hybridized. The chain extension product sequence that is produced is complementary to the template sequence. Usually, chain extension is enzyme catalyzed, preferably, in the present invention, by a thermostable DNA polymerase, such as the enzymes derived from Thermis acquaticus (the Taq polymerase), Thermococcus litoralis, and Pyrococcus furiosis.

Two nucleic acid sequences are “related” or “correspond” when they are either (1) identical to each other, or (2) would be identical were it not for some difference in sequence that distinguishes the two nucleic acid sequences from each other. The difference can be a substitution, deletion or insertion of any single nucleotide or a series of nucleotides within a sequence. Such difference is referred to herein as the “difference between two related nucleic acid sequences.” Frequently, related nucleic acid sequences differ from each other by a single nucleotide. Related nucleic acid sequences typically contain at least 15 identical nucleotides at each end but have different lengths or have intervening sequences that differ by at least one nucleotide.

The term “mutation” refers to a change in the sequence of nucleotides of a normally conserved nucleic acid sequence resulting in the formation of a mutant as differentiated from the normal (unaltered) or wild type sequence. Mutations can generally be divided into two general classes, namely, base-pair substitutions and frame-shift mutations. The latter entail the insertion or deletion of one to several nucleotide pairs. A difference of one nucleotide can be significant as to phenotypic normality or abnormality as in the case of, for example, sickle cell anemia.

A “duplex” is a double stranded nucleic acid sequence comprising two complementary sequences annealed to one another. A “partial duplex” is a double stranded nucleic acid sequence wherein a section of one of the strands is complementary to the other strand and can anneal to form a partial duplex, but the full lengths of the strands are not complementary, resulting in a single-stranded polynucleotide tail at least one end of the partial duplex.

The terms “hybridization,” “binding” and “annealing,” in the context of polynucleotide sequences, are used interchangeably herein. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is typically achieved by elevating the temperature, increasing the ratio of cosolvents, lowering the salt concentration, and other such methods well known in the field.

Two sequences are “complementary” when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence.

As used herein, a “single nucleotide polymorphism” or “SNP” refers to polynucleotide that differs from another polynucleotide by a single nucleotide exchange. For example, without limitation, exchanging one A for one C, G or T in the entire sequence of polynucleotide constitutes a SNP. Of course, it is possible to have more than one SNP in a particular polynucleotide. For example, at one locus in a polynucleotide, a C may be exchanged for a T, at another locus a G may be exchanged for an A and so on. When referring to SNPs, the polynucleotide is most often DNA and the SNP is one that usually results in a deleterious change in the genotype of the organism in which the SNP occurs.

By “being suspected of containing a polymorphism” is meant that the polynucleotide, usually DNA or RNA, being subjected to the method of this invention is one of known sequence, that sequence being known to be capable of containing a particular polymorphism at a known locus in the sequence.

As used herein, a “template” refers to a target polynucleotide strand, for example, without limitation, an unmodified naturally-occurring DNA strand, which a polymerase uses as a means of recognizing which nucleotide it should next incorporate into a growing strand to polymerize the complement of the naturally-occurring strand. Such DNA strand may be single-stranded or it may be part of a double-stranded DNA template. In applications of the present invention requiring repeated cycles of polymerization, e.g., the polymerase chain reaction (PCR), the template strand itself may become modified by incorporation of modified nucleotides, yet still serve as a template for a polymerase to synthesize additional polynucleotides.

As used herein, a “label” or “tag” refers to a molecule that, when appended by, for example, without limitation, covalent bonding or hybridization, to another molecule, for example, also without limitation, a polynucleotide or polynucleotide fragment, provides or enhances a means of detecting the other molecule. A fluorescence or fluorescent label or tag emits detectable light at a particular wavelength when excited at a different wavelength. A radiolabel or radioactive tag emits radioactive particles detectable with an instrument such as, without limitation, a scintillation counter.

A molecule that absorbs light at one wavelength and then emits detectable light at a second wavelength comprises a fluorescent label as defined above and is referred to herein as a “fluorophore.”

A “mass-modified” nucleotide is a nucleotide in which an atom or chemical substituents has been added, deleted or substituted but such addition, deletion or substitution does not create modified nucleotide properties, as defined herein, in the nucleotide; i.e., the only effect of the addition, deletion or substitution is to modify the mass of the nucleotide.

### Embodiments

One embodiment provides a method for detecting a polymorphism in a polynucleotide. The method includes annealing a probe to a polynucleotide to a region of the polynucleotide suspected of containing a polymorphism to form a complex, wherein the probe comprises a non-extendable 3′ end and is not complementary to the polymorphism. Generally, the probe anneals to the polynucleotide so that the polymorphism is between the 3′ and the 5′ end of the probe. The polymorphism can be 1, 2, 3, 4, 5, 6, or more consecutive or non-consecutive nucleotides. When the probe anneals to a polynucleotide having a polymorphism, a variation structure or a mismatch structure is produced. The structure can be a bulge, loop, or other configuration resulting from the mismatch of nucleotides between the probe and the polymorphism.

The method further includes contacting the complex with an enzyme or chemical that cleaves the probe and the polynucleotide at a region of mismatch between the probe and the polynucleotide to produce a probe with an extendible 3′ end. An artificial template is added wherein the cleaved probe acts as a primer for amplifying the artificial template. The method further includes amplifying the artificial template, wherein the presence of an amplified product indicates the presence of the polymorphism.

Another embodiment provides a method to detect a nucleotide base variation in a nucleic acid comprising (1) preparing a gene specific nucleic acid probe (probe) with non-extendable 3′ end; (2) hybridizing the probe to a target nucleic acid to form a duplex; (3) exposing the duplex to a cleavage enzyme or chemicals, wherein the enzyme or chemicals are able to recognize and cleave a structure resulting from a mismatch between the probe and the target nucleic acid; (4) cleaving the structure resulting from the mismatch to remove the non-extendable 3′ end from the probe and generate a new extendable 3′ end on the probe and optionally, on the target nucleic acid; (5) using the cleaved probe or target nucleic acid as a primer or/and template for selectively amplification by primer based or polymerase promoter based amplification methods; and (6) detecting amplified nucleic acid product, wherein the amplified product indicates the presence of a sequence variation or polymorphism in the target nucleic acid.

Target Nucleic Acid

The target nucleic acid or polynucleotide can be natural or synthetic DNA, RNA, or DNA-RNA hybrid in vitro or in vivo. The polynucleotide can be single-stranded or double-stranded. Typically the polynucleotide corresponds to a gene suspected of having a polymorphism at a predetermine location, for example a single nucleotide polymorphism. The polymorphism can be the result of a deletion, insertion, or substitution. The polymorphism is characterized relative to a known sequence, for example of a first allele. Thus, if the nucleotide sequence of the first allele is known, variations from that sequence, or polymorphisms can be detected. It is generally accepted that a singly polymorphism can give rise to a pathology, for example sickle cell anemia. The disclosed methods and compositions can therefore be used to detect or diagnose the presence or predisposition of a patient for a pathology related to a known polymorphism.

Gene Specific Probe

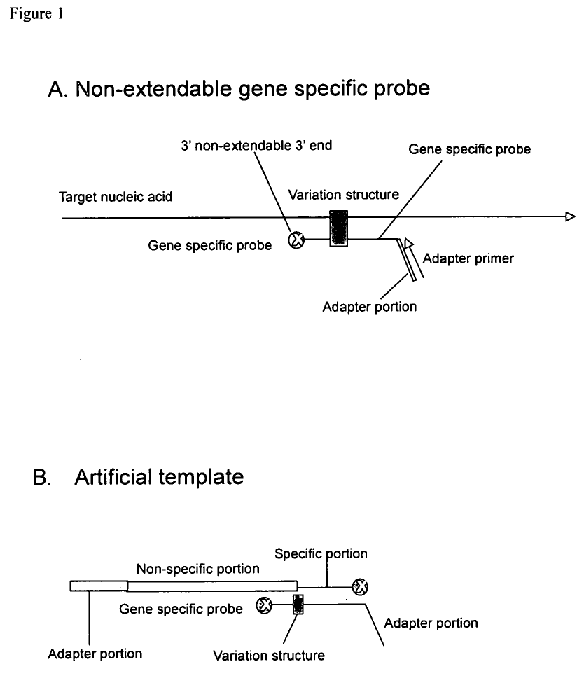

An non-extendable gene specific probe (probe) includes a sequence specific portion with an non-extendable 3′ end and optionally an adapter portion at 5′ end (FIG. 1). The sequence specific portion has a sequence complementary to a specific region of target nucleic acid, for example a region comprising a polymorphism. The sequence specific portion of the probe can be complementary to wild type or variant target nucleic acid at the specific region of interest. The gene specific probe optionally contains an adapter sequence that is not complementary to the target nucleic acid. This adapter sequence can include a sequence complementary to a promoter of RNA polymerase such as T7, T3 or SP6 promoter for future amplification. The gene specific probe can be an in vitro or in vivo synthesized nucleic acid including to DNA, RNA, or a combination thereof. The 3′ end of the gene specific probe is modified to be non-extendable to prevent extension reaction by polymerase. The modification can be achieved by adding a moiety that blocks the primer extension reaction. Blocking moieties include, but are not limited to chemical groups such as terminator nucleotides and nucleotide analogues, extra un-matched nucleotides, modified nucleotides, or a protein moiety (FIG. 1).

Artificial Template

An artificial template can also be used with the disclosed methods. The artificial template is a polynucleotide comprising a gene specific portion at 3′ end and a non-specific portion at the 5′ end. The 3′ end of the template is modified to block the extension by polymerase. The modification can be achieved by adding moiety that blocks the primer extension reaction include but not limited to chemical groups such as terminator nucleotides and nucleotide analogues, extra un-matched nucleotides, modified nucleotides, and a protein moiety. The gene specific portion has sequence complementary to the 3′ end portion of cleaved gene specific probe or target nucleic acid. The non-specific portion is not complementary to the gene specific probe or target nucleic acid. The non-specific portion optionally contains an adapter sequence complementary to the adapter primer or a sequence complementary to promoter sequence of RNA polymerase (FIG. 1B).

Adapter Primer

An adapter primer (adapter) has a nucleotide sequence complementary to the adapter portion of the gene specific probe (FIGS. 1A and B).

The Cleavage-Amplification Reaction

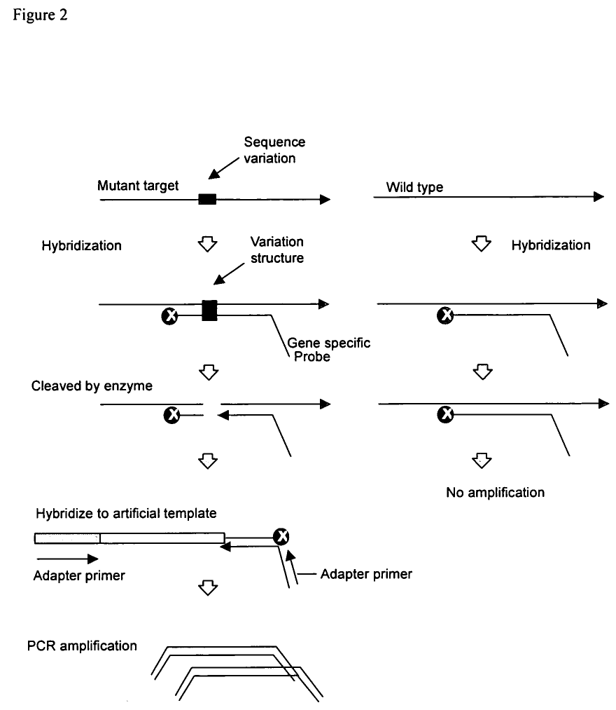

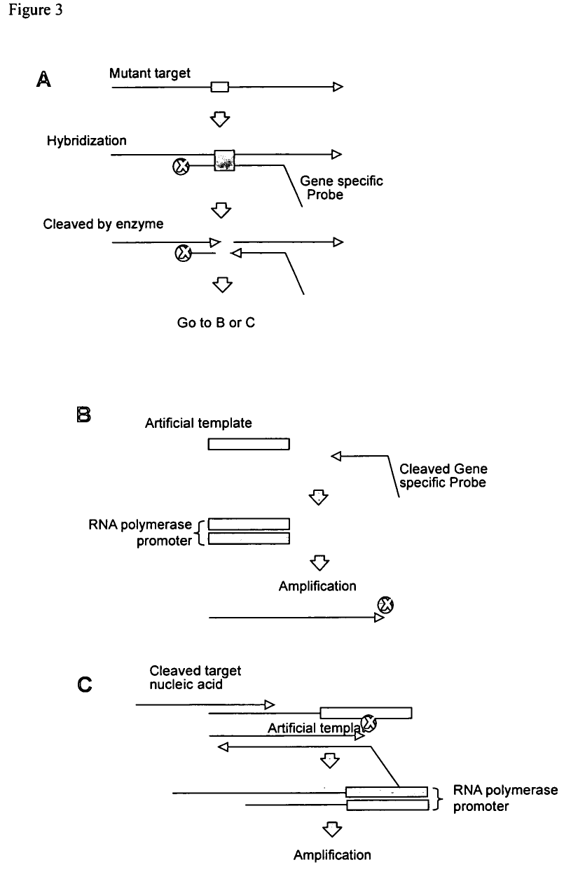

The target nucleic acid or polynucleotide suspected of having a polymorphism is mixed with the gene specific probe. The gene specific probe is designed to be complementary to the polynucleotide without a polymorphism. The mixture is heated to denature the nucleic acid, and then cooled to allow the probe to anneal with the target nucleic acid to form a duplex (FIG. 2). If the target nucleic acid has sequence variations or polymorphisms, the probe and the target nucleic acid form a variation structure or a mismatch structure in the duplex. The variation structure can be a newly generated restriction enzyme site created by the sequence variation or mismatched base pairs. The duplex is exposed to a reaction solution containing a cleavage enzyme or chemicals to chemically cleaves the variation structure in the duplex. The enzyme cleaves the variation structure thereby removing the non-extendable 3′ end of the probe to generated a new extendable 3′ end of the probe. The probe is activated and becomes extendable. The cleavage also generates a new extendable 3′ end on the target nucleic acid. The cleaved probe and target nucleic acid serve as a primer for primer based amplification or RNA polymerase promoter based amplification. Detection of any amplified nucleic acid indicates the presence of variation or polymorphism in the target nucleic acid (FIG. 2 and FIG. 3).

The primer based amplification methods included but not limited to PCR, strand displacement amplification, rolling circle amplification, and isothermal nucleic acid amplification (WO2004067726A2, WO2004059005).

For the PCR amplification, the cleaved gene specific probe or target nucleic acid serve as primers and the PCR amplification is performed between the cleaved gene specific probe and the artificial template (FIG. 2).

In another embodiment, the newly generated 3′ end of target nucleic acid or probe can extend with an artificial template containing a sequence complementary to a promoter of RNA polymerase to form a promoter structure. The cleavage signal can be detected by RNA polymerase amplification (FIG. 3).

In another embodiment, the newly generated 3′ end of target nucleic acid can extend with an un-cleaved probe or artificial template contain a sequence complementary to a promoter of RNA polymerase to form a promoter structure. The cleavage signal can be detected by RNA polymerase amplification (FIG. 3) To detect the amplification product, the gene specific probe, and the adapter primer can be tagged, hybridized with, or otherwise incorporate a detectable moiety or label. The moiety can be any type of detectable molecules includes but not limited to a fluorophore, biotin, digoxygenin, proteins such as protein tag, or antibody.

The gene specific probe, the gene specific reverse primer and the adapter primer can be immobilized to a solid phase or support for the purpose of separation and detection.

The cleavage enzyme used for cleaving the variation structure can be any type of restriction endonuclease and endonuclease that recognizes and cleaves all type of mismatches. Such enzymes include but not limited to bacteriophage T4 Endonuclease VII (Kosak et al., (1990) Eur. J. Biochem. 194: 779) or bacteriophage T7 Endonuclease I (deMassy, B., et al. (1987) J. Mol. Biol. 193: 359), S1 nuclease, Mung bean nuclease. Mut Y, Mut H, Mut S and Mut L repair protein family (Welsh, K. M. et al (1987) J. Biol. Chem. 262, 15624-15629), CEL nuclease family of mismatch nucleases derived from celery (Oleykowski, C. A. at al (1998). Nuc. Acids Res. 26:4597-4602).

The mismatch structure also can be cleaved by treatment with chemicals such as hydroxylamine or osmium tetroxide.

The amplified nucleic acid can be detected by measuring UV absorbance or by staining with a detectable dye such as fluorescent dye cyber green. The detection also can be achieved by labeling the amplification product using a labeled probe, primer, or incorporate a labeled nucleotide into the amplification product. The amplified product can be detected by measure the pyrophosphate (PPi) generated from the amplification reaction. The methods can utilize size fractional approach such as gel electrophoresis, capillary electrophoresis, HPLC, and mass spectrometer can be use or combined with labeling methods for the detection.

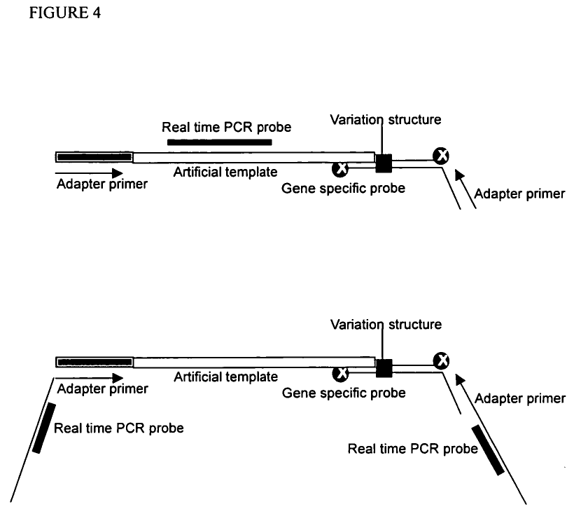

The amplification also can be performed with real time PCR. A labeled probe is designed for real time PCR hybridization to any portion of the adapter sequence in the gene specific probe and adapter primer, or the sequence between the probe and primers (FIG. 4). The labeled probe includes but not limited to Taqman probe, Molecular beacon probe, or a Scopine probe.

Another embodiment provides a kit comprising a probe designed for detecting a specific nucleic acid polymorphism, an adapter primer, an artificial template, and an enzyme or chemical for cleaving a mismatch structure. The kit optionally includes reagents for amplifying the artificial template and instructions for using the kit to detect a specific polymorphism.

Detectable Labels

The disclosed probes or targets can include a detectable label, for example, a first detectable label. Sample polynucleotides can include a detectable label, for example, a second detectable label. Suitable labels include radioactive labels and non-radioactive labels, directly detectable and indirectly detectable labels, and the like. Directly detectable labels provide a directly detectable signal without interaction with one or more additional chemical agents. Suitable of directly detectable labels include colorimetric labels, fluorescent labels, and the like. Indirectly detectable labels interact with one or more additional members to provide a detectable signal. Suitable indirect labels include a ligand for a labeled antibody and the like.

Suitable fluorescent labels include any of the variety of fluorescent labels known in the art. Specific suitable fluorescent labels include: xanthene dyes, e.g., fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g., Cy3, Cy5 and Cy7 dyes; coumarins, e.g., umbelliferone; benzimide dyes, e.g., Hoechst 33258; phenanthridine dyes, e.g., Texas Red; ethidium dyes; acridine dyes; carbazole dyes; phenoxazine dyes; porphyrin dyes; polymethine dyes, e.g., cyanine dyes such as Cy3, Cy5, etc; BODIPY dyes and quinoline dyes.

### EXAMPLES

### Example 1

Identification of the K-ras point mutation in codon 12 (GGT>GAT)

The K-ras mutation in codon 12 (GGT>GAT) creates a new restriction enzyme site for BccI. To detect the mutation, a probe complementary to the flanking sequence at both side of the mutation is designed for the cleavage and amplification.

The Non-Extendable Gene Specific Probe

(SEQ ID NO: 1)

5′-TGTTCTTGTTTATTCGACACAGTTCTTCATAAACTTGTGGTAGTTGG
AGCTGATGGTTT*

*is inverted dTTP

The Artificial Template

(SEQ ID NO: 2)

5′-CTTGTTCTTGTTTATTCGACACAGTTCTTC GCTTTGGCCG
CCGCCCAGTC CTGCTCGCTT CGCTACTTGG AGCCACTATC
GACTACGCGA TCATGGCGAC CACACCCGTC CTGTGGATCC
TCTACGCCGG ACGCATCGTG GCTCCAACTACCACAAGTTTA
TCCGAAA*.

*is ddATP

The Adapter Primer

| | |
|---|---|
| CTTGTTTATTCGACACAGTTCTTC | (SEQUENCE ID NO: 3) |

The DNA Samples

The wild type genome DNA is purchased from Promega and Mutant DNA was extracted from human pancreas adenocarcinoma cells from ATCC (#CRL-2547) by using commercial DNA extraction kit (Qiagen). The final concentration of genomic DNA was adjusted to 100 ng/ul.

The Hybridization

The hybridization was performed in a total 10 ul of hybridization solution contains 0.1-1 ug genome DNA, 0.05 uM of the probe, 10 mM Tris-HCl, pH 7.0, 10 mM NaCl. The mixture was heated at 95° C. for 5 minutes and then cooled down to 50° C. for 25 minutes.

The Enzyme Cleavage

The cleavage was performed in total 10 ul solution containing 10 mM Tris-HCl, pH 7.0, 10 mM MgCl₂, 1 mM dithiothreitol, 100 μg/ml Bovine Serum Albumin, and 2 units of BccI (New England BioLab) at 37° C. for 1 hour.

The Amplification

After the cleavage, 10 ul of cleavage mixture was transferred to 40 ul of the amplification solution containing a final concentration of 0.1 uM artificial template (SEQ ID NO: 2), 0.5 uM of adapter primer (SEQ ID NO: 3), 0.2 mM of dATP, dCTP, dGTP, and dTTP, 20 mM Tris-HCl, pH 8.8, 15 mM (NH4)₂SO₄, 1.5 mM MgCl₂, 2 units of platinum Taq polymerase (Invitrogen). The PCR amplification was performed in a thermal cycler (Hybaid), the cycle condition was as follows: 1 cycle of 95° C. for 5 minutes, 35 cycles of 95° C. for 1 min, 56° C. for 1 minutes, 72° C. for 1 minutes. 1 cycle of 72° C. for 10 minutes. After PCR reaction, 10 ul of PCR product was analyzed on 1.2% agarose gels and the DNA band was visualized by staining with ethidium bromide.

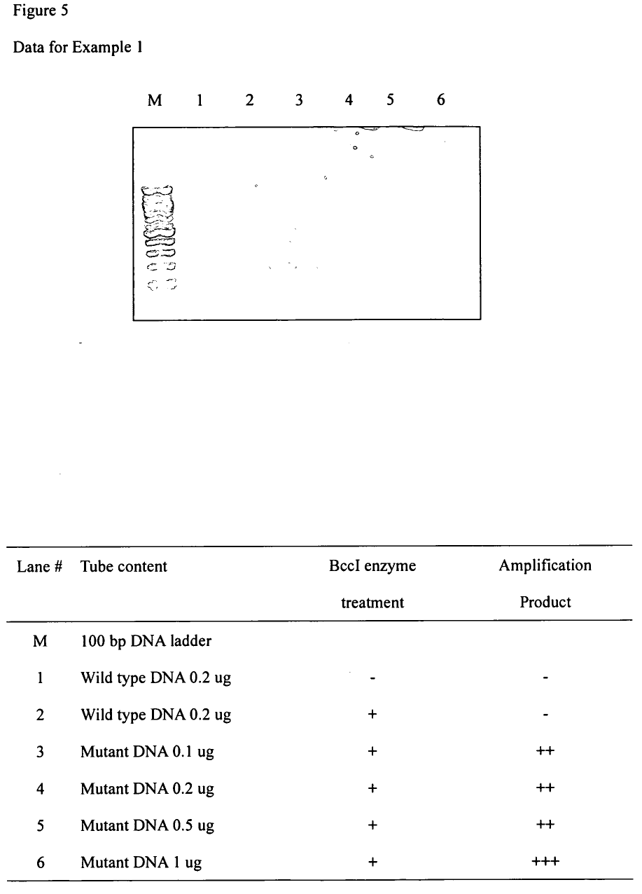

The results are shown in FIG. 5 and summarized in the table below

| | | BccI enzyme | Amplification |
|---|---|---|---|
| Lane # | Tube content | treatment | Product |
| M | 100 bp DNA ladder | | |
| 1 | Wild type DNA 0.2 ug | − | − |
| 2 | Wild type DNA 0.2 ug | + | − |
| 3 | Mutant DNA 0.1 ug | + | ++ |
| 4 | Mutant DNA 0.2 ug | + | ++ |
| 5 | Mutant DNA 0.5 ug | + | ++ |
| 6 | Mutant DNA 1 ug | + | +++ |

A 198 base pair PCR amplified product was detected in the tube containing mutant DNA. No amplification product was detected using wild type DNA (FIG. 5). No amplification shown in the control tubes without the DNA template or with DNA template without enzyme treatment (FIG. 5). The amplified PCR product indicated the presence of mutation allele in the sample.

### Example 2

Identification of the B-raf Mutation in Codon 599 (GTG>GAG)

The B-raf mutation in codon 599 (GTG>GAG) does not created a new site for a restriction enzyme. To detect the mutation, a complementary probe with wild type sequence is designed to hybridize and form a mismatch structure with mutant allele. The probe will be cleaved at the mismatch position by a mismatch cleavage enzyme. The cleaved probe will serve as a primer for PCR amplification.

The Non-Extendable Gene Specific Probe

(SEQ ID NO: 4)

5′-GTTCTTGTTTATTCGACACAGTTCTTCGGTGATTTTGGTCTAGCTAC
AGTGAAATCTC*A*G*T*T*T**

*is a nucleotide base with thiol modifier
**is inverted dTTP.

The Artificial Template

(SEQ ID NO: 5)

5′-CTTGTTCTTGTTTATTCGACACAGTTCTTC GCTTTGGCCG
CCGCCCAGTC CTGCTCGCTT CGCTACTTGG AGCCACTATC
GACTACGCGA TCATGGCGAC CACACCCGTC CTGTGGATCC
TCTACGCCGG ACGCATCGTG CATTTCACTGTAGCTAGACCA
AAATCACCTTTT*

*is ddTTP

The DNA Template

Genomic DNA is prepared from thyroid cancer cell line as described in the J. Clinical Endocrinology & Metabolism 89(6):2867-2872. The final concentration of genomic DNA is adjusted to 100 ng/ul.

The Hybridization

The hybridization is performed in total 20 ul solution contains 1 ug of genome DNA, 0.05 uM probe (SEQ ID NO: 4), 20 mM Tris-HCl (pH 8.0), 50 mM NaCl. The mixture was heated at 95° C. for 5 minutes and then incubated at 50° C. for 25 minutes.

The Enzyme Cleavage

After hybridization, 2 units of Cel I nuclease (Transgenomic Inc) were added to the hybridization mixture. The cleavage was performed at 37° C. for 1 hour. After cleavage the enzymes were heat-inactivated in the tube (95° C. for 10 minutes).

The Amplification

PCR amplification and the results analysis were performed under the conditions described in Example 1. The final concentration for artificial template (SEQ ID 5) is 0.1 uM and for adapter primer (SEQ ID 3) is 0.5 uM.

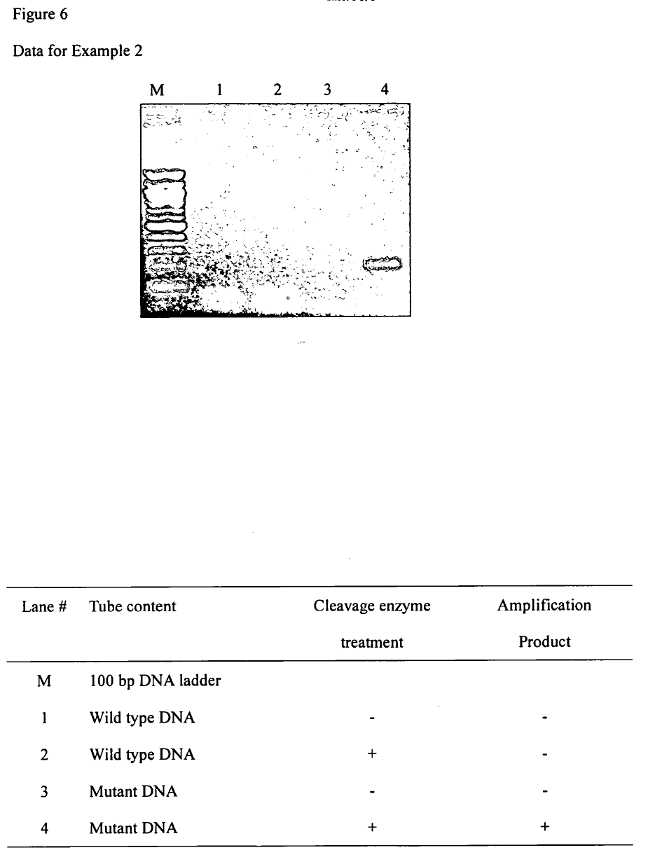

The results are shown in FIG. 6 and summarized in the table below

| | | Cleavage enzyme | Amplification |
|---|---|---|---|
| Tube # | Tube content | treatment | Product |
| 1 | Wild type DNA | − | − |
| 2 | Wild type DNA | + | − |
| 3 | Mutant DNA | − | − |
| 4 | Mutant DNA | + | + |

A specific 200 base pair amplification product was detected from the tube #4 containing probe and DNA with mutation but not from the tube #2 containing the probe and wild type DNA sample. The results indicated the presence of mutation in the sample.

1. A method for detecting a polymorphism in a polynucleotide, comprising:
(a) annealing a probe to a region of a polynucleotide suspected of containing a polymorphism to form a complex, wherein the probe comprises a non-extendable 3′ end and is not complementary to the polymorphism;
(b) contacting the complex with an enzyme or chemical that cleaves the probe and the polynucleotide at a region of mismatch between the probe and the polynucleotide to produce a probe with an extendible 3′ end;
(c) adding an artificial template, wherein the cleaved probe acts as a primer for amplifying the artificial template; and
(d) amplifying the artificial template, wherein the presence of an amplified product indicates the presence of the polymorphism.

2. The method of claim 1, wherein said the region of mismatch between the probe and the polynucleotide includes a newly generated restriction enzyme site.

3. The method of claim 1, wherein said the target nucleic acid is obtained from a nature source or in vitro or in vivo synthesized nucleic acid.

4. The method of claim 1, wherein said probe is in vitro or in vivo synthesized DNA, RNA, or a chimera of DNA and RNA.

5. The method of claim 1, wherein said probe comprises an adapter sequence at its 5′ end, wherein the adapter sequence is not complementary to the polynucleotide.

6. The method of claim 5, wherein said the adapter sequence of the probe comprises a sequence complementary to a promoter of RNA polymerase.

7. The method of claim 6, wherein the RNA polymerase is selected from the group consisting of T7, T3 and SP6 polymerase.

8. The method of claim 1, wherein the enzyme is a restriction endonuclease.

9. The method of claim 1, wherein said the enzyme is an endonuclease.

10. The method of claim 9, wherein the enzyme is selected from the group consisting of bacteriophage T4 Endonuclease VII, bacteriophage T7 Endonuclease I, S1 nuclease, Mung bean nuclease, Mut Y, Mut H, Mut S, Mut L, and CEL nuclease family.

11. The method of claim 1, wherein the chemical is selected from the group consisting of hydroxylamine and osmium tetroxide.

12. The method of claim 1, wherein extension and amplification is performed by DNA polymerase with or without strand displacement activity.

13. The method of claim 1, wherein said amplification is performed using a method selected from the group consisting of PCR, strand displacement amplification, rolling circle amplification, and isothermal nucleic acid amplification method.

14. The method of claim 1, wherein said the artificial template comprises a non-extendable 3′ end.

15. The method of claim 14, wherein the artificial template comprises a 3′ region complementary to the polynucleotide and a non-specific region at a 5′ end.

16. The method of claim 15, wherein the non-specific region comprises an adapter complementary to an adapter primer or promoter sequence of an RNA polymerase.

17. The method of claim 1, wherein the non-extendable 3′ end of the probe is modified to block extension by DNA polymerase.

18. The method of claim 1, wherein the artificial template comprises a sequence complementary to a promoter of RNA polymerase.

19. The method of claim 1, wherein amplified template is detected by measuring UV absorbance.

20. The method of claim 1, wherein the amplified template comprises a labeled nucleotide.

21. The method of claim 1, wherein amplification is detected by measuring pyrophosphate generated from an amplification reaction.

22. The method of claim 1, wherein s amplification is detected using gel electrophoresis, capillary electrophoresis, HPLC, or mass spectrometry.

23. The method of claim 20, wherein the label is selected from the group consisting of a fluorophore, biotin, digoxygenin, a protein tag, antibody, and an enzyme conjugate.

24. The method of claim 1, wherein the probe, the artificial template and optionally, an adapter primer are immobilized on a solid support.

25. The method of claim 5, wherein the amplification is performed by real time PCR with a labeled probe designed for any portion of the adapter sequence.

26. A method for detecting nucleotide variations between target nucleic acid comprising:
(a) preparing a gene specific probe with an non-extendable 3′ end, wherein the probe is complementary to a region of the target nucleic acid;
(b) hybridizing the gene specific probe to the target nucleic acid to form a duplex, wherein a variation structure is formed in the duplex if the target nucleic acid comprises a nucleotide variation;
(c) exposing the duplex to a cleavage enzyme or chemicals, wherein the enzyme or the chemicals cleave the variation structure in the duplex to remove the non-extendable 3′ end from the gene specific probe and generated a new extendable 3′ end on the probe and the target nucleic acid; and
(d) amplifying an artificial template using the cleaved gene specific probe or target nucleic acid as primers.

27. The method of claim 26, wherein amplifying occurs by RNA polymerase promoter based amplification.


SEQUENCE LISTING (5 sequences)

SEQ ID NO: 1 - DNA, 59 bp, Artificial Sequence
  artificial probe sequence
tgttcttgtttattcgacacagttcttcataaacttgtggtagttggagctgatggttt

SEQ ID NO: 2 - DNA, 168 bp, Artificial Sequence
  artificial template sequence
cttgttcttgtttattcgacacagttcttcgctttggccgccgcccagtcctgctcgcttcgctacttggagccactatcgactacgcgatcatggcgaccacacccgtcctgtggatcctctacgccggacgcatcgtggctccaactaccacaagtttatccgaaa

SEQ ID NO: 3 - DNA, 24 bp, Artificial Sequence
  adapter primer
cttgtttattcgacacagttcttc

SEQ ID NO: 4 - DNA, 63 bp, Artificial Sequence
  artificial probe sequence
gttcttgtttattcgacacagttcttcggtgattttggtctagctacagtgaaatctcagttt

SEQ ID NO: 5 - DNA, 173 bp, Artificial Sequence
  artificial template sequence
cttgttcttgtttattcgacacagttcttcgctttggccgccgcccagtcctgctcgcttcgctacttggagccactatcgactacgcgatcatggcgaccacacccgtcctgtggatcctctacgccggacgcatcgtgcatttcactgtagctagaccaaaatcacctttt